(19) United States
(12) Patent Application Publication
(10) Pub. No.: US 2022/0195395 A1
(43) Pub. Date: Jun. 23, 2022

(54) PHYSIOLOGIC GROWTH OF CULTURED INTESTINAL TISSUE

(71) Applicant: The Regents of the University of Michigan, Ann Arbor, MI (US)

(72) Inventors: Charlie Childs, Ann Arbor, MI (US); Emily Holloway, Ann Arbor, MI (US); Jason Spence, Ann Arbor, MI (US)

(21) Appl. No.: 17/549,028

(22) Filed: Dec. 13, 2021

Related U.S. Application Data
(60) Provisional application No. 63/126,581, filed on Dec. 17, 2020.

(51) Int. Cl. C12N 5/071; G01N 33/50

(52) U.S. Cl. CPC C12N 5/0679; G01N 33/5014; G01N 33/5044; C12N 2506/45; C12N 2501/415; C12N 2501/119; C12N 2533/90; C12N 2501/155

(57) ABSTRACT

The invention disclosed herein generally relates to methods and systems for improving physiological growth of cultured tissues. In particular, the invention disclosed herein relates to methods and systems for promoting maintenance of cultured intestinal organoids (e.g., derived from pluripotent stem cells or from primary sources such as biopsy tissue).

### STATEMENT OF RELATED APPLICATIONS

This application claims priority to U.S. Provisional Patent Application No. 63/126,581, filed Dec. 17, 2020, the entire contents of which are incorporated herein by reference for all purposes.

### STATEMENT REGARDING FEDERALLY SPONSORED RESEARCH

This invention was made with government support under DK103141 awarded by the National Institutes of Health. The government has certain rights in the invention.

### FIELD OF THE INVENTION

The invention disclosed herein generally relates to methods and systems for improving physiological growth of cultured tissues. In particular, the invention disclosed herein relates to methods and systems for promoting maintenance of cultured intestinal organoids (e.g., derived from pluripotent stem cells or from primary sources such as biopsy tissue).

### BACKGROUND

The stem cell niche within a tissue is required to regulate stem cell maintenance, self-renewal and differentiation (Scadden, Nature 441, 1075-1079 2006). The niche is made up of both physical and chemical cues, including the extracellular matrix (ECM), cell-cell contacts, growth factors and other small molecules such as metabolites (Capeling et al., Stem Cell Reports 12, 381-394 2019; Cruz-Acuña et al., Nat. Cell Biol. 2017; Gjorevski et al., Nature 539, 560-564 2016). Understanding the niche within various tissues has been central to understanding how tissues maintain homeostasis, and for understanding how disease may occur (Van de Wetering et al., Cell 111, 241-250 2002). Further, establishing proper in vitro niche conditions has allowed the growth and expansion of gastrointestinal tissue-derived stem cells in culture (Dedhia et al., Gastroenterology 150, 1098-1112 2016; Kretzschmar and Clevers, Dev. Cell 38, 590-600 2016). For example, through understanding that WNT signaling is important for maintaining intestinal stem cell (ISC) homeostasis (Muncan et al., Mol Cell Biol 26, 8418-8426 2006; Pinto et al., Genes 2003; Sansom et al., Genes Dev. 18, 1385-1390 2004), blockade of BMP signaling by NOGGIN (NOG) promotes ectopic crypt formation (Haramis et al., Science (80) 303, 1684-1686 2004), and that EGF is a potent stimulator of proliferation (Goodlad et al., Gut 28 Suppl, 37-43 1987; Ulshen et al., Gastroenterology 91, 1134-1140 1986), it was determined that WNTs, RSPONDINs (RSPOs), NOG and EGF can be utilized to expand and maintain ISCs in culture as 3-dimensional intestinal organoids (Ootani et al., Nat. Med. 15, 701-706 2009; Sato et al., Nature 459, 262-265 2009, Gastroenterology 141, 1762-1772 2011). This same information has been leveraged to expand and culture human pluripotent stem cell derived intestinal organoids in vitro (Finkbeiner et al., Stem Cell Reports 4, 1140-1155 2015; Spence et al., Nature 470, 105-109 2011; Wells and Spence, Development 141, 752-760 2014).

Despite significant progress over the past decade, it is also clear that current in vitro systems are still not optimized to most accurately reflect the in vivo environment. Ongoing efforts are aimed at improving the in vitro physical environment through biomimetic ECM (Capeling et al., Stem Cell Reports 12, 381-394 2019; Cruz-Acuña et al., 2017, supra; Gjorevski et al., Nature 539, 560-564 2016), and by adjusting signaling cues to more accurately reflect the in vivo niche (Fujii et al., Cell Stem Cell 23, 787-793.e6 2018). More recently, single cell technologies have started to reveal unprecedented amounts of information about the cellular heterogeneity of human intestinal tissue and the ISC niche during health and disease (Kinchen et al., Cell 175, 372-386 2018; Martin et al., Cell 178 2019; Smillie et al., Cell 178, 714-730.e22 2019), and will undoubtedly yield substantial information about cell types and niche cues that regulate ISCs in various contexts.

Additional factors for culture, maintenance, and differentiation of organoids in vitro are needed.

### SUMMARY

Experiments described herein demonstrated that the human fetal intestinal stem cell niche is composed of multiple cellular sources, and highlighted a unique role for different ligands from the EGF family. The systems and methods described herein utilize such factors to provide robust and physiologic culture conditions for generating and maintaining intestinal organoids.

For example, in some embodiments, provided herein is a method of culturing intestinal cells or tissue, comprising: culturing intestinal cells or tissue (e.g., human intestinal cells or tissue) in vitro, wherein the culturing comprises culturing in culture medium comprising neuregulin 1 (NRG1) or epiregulin (EREG).

The present disclosure is not limited to particular intestinal tissue or cells. In some embodiments, the cells are stem cells (e.g., pluripotent stem cells such as iPSCs). In some embodiments, the intestinal tissue is fetal intestine.

In some embodiments, the culturing results in the formation of intestinal epithelial organoids, which are commonly referred to as enteroids. In some embodiments, the culturing results in stem cell maintenance of said intestinal cells or tissue. In some embodiments, the culturing is in Matrigel. In some embodiments, the culture medium further comprises R-spondin (e.g., R-spondin 1, 2, or 3) and Noggin. In some embodiments, the culture medium does or does not comprises EGF. In some embodiments, the culture medium further comprises Epiregulin (EREG). In some embodiments, the intestinal cells or tissue exhibit maintenance of intestinal cells or tissue cultured in culture medium comprising NRG1 or EREG.

Further embodiments provide compositions, systems, or kits comprising human intestinal organoids obtained through the methods described herein.

Additional embodiments provide a method of screening a compound, comprising: a) contacting an intestinal organoid described herein with a test compound (e.g., drug); and b) assaying the effect of the test compound on one or more properties of the intestinal enteroid (e.g., proliferation, toxicity, and the like).

Also provided is the use of an intestinal organoid obtained through a method described herein to treat or prevent an intestinal disease or condition.

Further provided is a method of treating an intestinal disease or condition, comprising: implanting an intestinal organoid obtained through a method described herein in the intestine of a subject in need thereof.

Additional embodiments are described herein.

### BRIEF DESCRIPTION OF THE DRAWINGS

FIG. 1. Mesenchymal heterogeneity in the developing human duodenum. (A) H&E staining on human fetal intestine sections at a constant scale spanning from 54 d to 130 d (days post conception) (B) Timeline of specimens and corresponding number of cells profiled by scRNA-seq after filtering and ‘cleaning’ of ambient/background RNA. (C) UMAP visualization of each sample analyzed by scRNA-seq displayed by the age post conception. (D) Following application of Harmony to mesenchymal cells from all time points, a force directed layout illustrates the relationship between timepoints. (E) Feature plots of individual genes for various lineages are shown, including PDGFRA, F3, DLL1, NPY, GPX3, TAGLN, ANO1, and RGS5 plotted onto the force-directed layout presented in FIG. 1C. (F) Representative images from FISH staining for F3 (pink) and immunofluorescent protein staining for SM22 (TAGLN protein product; green) with DAPI (grey) on the developing human intestine (n=1 biological replicate per timepoint) (G) Spatial characterization of PDGFRA^{HI}/DLL1^{HI}/F3^{HI} and GPX3^{HI} mesenchymal cells using FISH in the developing human intestine.

FIG. 2. Interrogating stem cell niche factors in the developing human intestine. (A) Summary schematic annotating the approximate expression domains of several mesenchymal subpopulations on the force directed layout as identified in FIG. 1E and FIG. 6C. (B) Feature plots of several individual ISC niche factors including EGF, 481 NRG1, WNT2B, RSPO2, and RSPO3 in mesenchymal cells at all time points profiled. (C) Multiplexed FISH for niche factors EGF (green), NRG1 (green), WNT2B (pink), RSPO2 (pink), and RSPO3 (pink) coupled with immunofluorescent protein staining of SM22 (blue), DAPI (grey), and FISH for F3 (green) in developing human fetal crypts. (D) Following application of Harmony to epithelial cells from all time points, force directed layout illustrates the relationship among timepoints. Cells are colored by sample identity (days post conception). (E) Feature plots of EGFR, ERBB2, ERBB3, and ERBB4 plotted onto the force directed layout presented in FIG. 2D. (F) FISH staining in developing human fetal crypts for EGFR (pink), ERBB2 (green), and ERBB3 (red) coupled with immunofluorescent staining for ECAD (blue) and DAPI (grey) (G) Feature plots of EGF and the enterocyte marker FABP2 plotted onto the force-directed layout presented in FIG. 2D. (H) Representative images of multiplexed FISH for EGF (pink) and NRG1 (green), coupled with immunofluorescent protein staining of MKI67 (blue) and DAPI (grey).

FIG. 3. NRG1 does not support proliferation and growth of established enteroids lines (A) Experimental schematic for data presented in 3A-I. (B) Representative stereomicroscope images after 5 days of growth in the presence of EGF (100 ng/ml) or NRG1 (100 ng/ml). (C) Representative images of FISH staining for OLFM4 (pink) or immunofluorescent protein staining for MKI67 (pink) coupled with ECAD (blue) and DAPI (grey) in enteroids grown in EGF (100 ng/ml) or NRG1 (100 ng/ml). (D) UMAP embedding of enteroid scRNAseq data (5,509 cells total) demonstrating the 5 precited clusters (n=1 biological sample sequenced, 142 d fetal sample). (E) UMAP embedding of enteroid scRNAseq data colored by culture condition (EGF-2,789 cells; NRG1-2,720 cells). (F) Feature plot of MKI67 illustrating that most proliferating cells are within cluster 4. (F) Bar chart depicting the percentage of cells in cluster 4 from each treatment group. (H) Feature plots demonstrating the expression of the stem cell marker OLFM4, secretory progenitor marker SPDEF, and enterocyte marker FABP1. (I) Experimental schematic for enteroid forming assays (left).

FIG. 4. Establishment of new enteroid lines in NRG1 increases cell type diversity in vitro. (A) Experimental schematic. Enteroids were established from 132 d (B-C) or 105 d (D-J) human fetal specimen in the presence of EGF (100 ng/ml), NRG1 (100 ng/ml), both EGF (100 ng/ml) and NRG1 (100 ng/ml), or without any EGF or NRG1. (B) Representative stereoscope images of enteroids in each condition 8 days after placing isolated epithelium in Matrigel with growth factors. Scalebars represent 1 mm (C) Representative images of FISH for OLFM4 (pink) or immunofluorescent protein staining for MKI67 (pink), MUC2 (pink), LYZ (pink) and ECAD (blue) and DAPI (grey) in enteroids after P0, 11 days of growth in the presence of EGF, NRG1, or both EGF and NRG1. (D) UMAP embeddings of 13,205 enteroid cells colored by cluster identity. (E) UMAP embeddings of enteroid cells colored by sample identity (EGF-3,262 cells, NRG1-7,350 cells, and EGF/NRG1-2,593 cells). (F) Bar charts depicting the cell type abundance (% of cells total sequenced) for each condition. (G) Dotplots for the proliferation markers MKI67 and TOP2A. (H) Bar chart depicting the proportion of cells sequenced that map to proliferative clusters (cluster 4 or 8) in each condition. (I) Feature plots for intestinal epithelial lineages include ISCs (OLFM4, clusters 0, 1, 2, 3, 4, 8), enterocytes (FABP2, SI—cluster 6), enteroendocrine cells (CHGA—cluster 12), and goblet cells (MUC2—cluster 11). (J) Bar chart depicting the proportion of cells sequenced for each condition are present in cluster 6 (enterocytes), cluster 12 (enteroendocrine cells), and cluster 11 (goblet cells).

FIG. 5. Mapping to an intestinal reference reveals cellular heterogeneity in enteroids. (A) Epithelial cells (828 cells) from primary intestine specimens (n=4; 101 d, 122 d, 127 d, 132 d) were computationally extracted, re-clustered, and visualized using UMAP. (B) Cluster identities were assigned based on expression of canonical lineage markers. (C) The Ingest function was used to map enteroids derived in the presence of EGF (100 ng/ml), NRG1 (100 ng/ml), or both EGF and NRG1 (100 ng/ml) onto to primary intestinal epithelium reference presented in 5A. (D) The abundance of cells mapping to each of the 9 clusters identified in the in vivo intestinal epithelium was determined for the primary intestinal epithelium and for enteroids in each treatment group.

FIG. 6. Cell cluster annotation of primary human fetal intestine specimens profiled by scRNA-seq. (A) Dotplot of canonical genes associated with major cell classes for each sample sequenced: Neurons (S100B, PLP1, STMN2, ELAVL4); endothelium (CDH5, KDR, ECSSR, CLDN5); mesenchyme (COL1A1, COL1A2, DCN, ACTA2, TAGLN, MYLK); epithelium (EPCAM, CDH1, CDX2, CLDN4); immune (PTPRC, HLA-DRA, ARHGDIB, CORO1A). Age is shown as days (d) post conception. (B) Mesenchymal cells from each timepoint were computationally extracted, re-clustered, and visualized using UMAP for each sample. (C) Feature plots of individual genes for various mesenchymal subpopulations are shown, including DLL1, PDGFRA, F3, NPY, GPX3, and TAGLN. (D) Feature plots of additional individual genes for various lineages are shown, including DCN, COL1A1, COL1A2, ACTA2, FRZB, SOX6 plotted onto the force-directed layout presented in FIG. 1D.

FIG. 7. Lower magnification imaging for spatial characterization of mesenchymal subpopulations and ISC niche factors. (A) Lower magnification image of multiplex FISH for F3 (pink), PDGFRA (green) and IF for ECAD (blue) in developing human intestine (representative data shown from n=2 biological replicates, 120 d fetal sample shown). (B) Multiplex FISH demonstrating co-expression of F3 (pink) and NRG1 (green) in developing human intestine (representative data shown from n=1 biological replicates of 132 d sample). (C) Multiplex FISH demonstrating co-expression of F3 (pink), FRZB (green) and IF for SM22 (blue) in developing human intestine (representative data shown from n=2 biological replicates, 132 d fetal sample shown). (D) Multiplex FISH for NPY (pink) and F3 (green) in developing human intestine (representative data shown from n=1 biological replicates of 132 d sample). (E) Multiplex FISH for DLL1 (pink) and F3 (green) in developing human intestine (representative data shown from n=1 biological replicates of 132 d sample). (F) Lower magnification image of multiplex FISH for RSPO2, RSPO3, and WNT2B (pink) and F3 (green) and IF for SM22 (blue) in developing human intestine (representative data shown from n=2 biological replicates, 120 d fetal sample shown). (G) Lower magnification image of multiplex FISH for EGFR (pink), ERBB2 (green), and ERBB3 (red) and IF for ECAD (blue) in developing human intestine. (H) Representative images of multiplex FISH using commercial negative control probes (Cy3 and Cy5 channels), and IF for SM22 (blue) in developing human intestine to demonstrate tissue autofluorescence of by red blood cells and epithelium.

FIG. 8. Investigation of EGF family ligand expression by scRNA-seq. (A) Feature plots displaying mesenchymal expression of additional EGF-family ligands NRG2, NRG3, NRG4, TGFA, HBEGF, AREG, BTC, EPGN, and EREG plotted onto the force-directed layout presented in FIG. 1D. (B) Feature plots from each developmental specimen for EGF, NRG1, and F3 interrogating expression in the entire scRNA-seq data set for each sample.

FIG. 9. Enteroids established in NRG1 can be passaged at similar efficiencies to those established in standard EGF conditions. (A) Stereomicroscope images at passage (P) P1, P2, P4, and P5 in EGF (100 ng/ml), NRG1 (100 ng/ml), or both EGF (100 ng/ml) and NRG1 (100 ng/ml). (B) Stereoscope images of enteroids after single-cell passaging 1,000 or 10,000 cells at P2 and growth in EGF (100 ng/ml), NRG1 (100 ng/ml), or both EGF (100 ng/ml) and NRG1 (100 ng/ml). (C) Quantification of number enteroids formed from 1,000 single cells.

FIG. 10. Epiregulin's Expression in the Developing Human Intestine (A) UMAP visualization of all sample analyzed by scRNA-seq. (B) UMAP visualization of the epithelial cluster (green cluster 2). (C) Dot plot of stem cell markers (LRG5 and OLFM4), all EGF family member ligands, and receptors expression levels across clusters found in B.

FIG. 11. Epiregulin creates putative crypt like structures in human enteroid cultures (A) Schematic demonstrating enteroid experiment parameters for single cell analysis. (B) Representative brightfield images of 100 ng/ml EGF, 100 ng/ml NRG1, 10 ng/ml EREG, and 1 ng/ml EREG enteroid cultures at passage 1 day 11. (C) UMAP Visualization of all four culture conditions of interest sample clustering. (D) Feature plots of stem cell (LRG5 and OLFM4), proliferation (MKi67), goblet cell (MUC2), enterocyte (SI), and enteroendocrine (CHGA) gene expression. (E) UMAP visualization of 1 ng/ml EREG grown enteroids with clusters including stem cells, goblet cells, enteroendocrine cells, enterocytes, and BEST4+ enterocytes. (F) Dot plot with canonically expressed genes used to annotate clusters in E. (G) FISH and IF of 1 ng/ml EREG enteroids showing spatial location of stem cells, proliferative cells, goblet cells, enteroendocrine cells, and differentiated cells.

### Definitions

As used herein, the term “pluripotent stem cells (PSCs),” also commonly known as PS cells or induced pluripotent stem cells (iPSCs), encompasses cells that can differentiate into any cell type found in the human body, i.e., cells derived from any of the three germ layers, including endoderm (interior stomach lining, gastrointestinal tract, the lungs, liver, pancreas), mesoderm (muscle, bone, blood, urogenital), and ectoderm (epidermal tissues and nervous system). PSCs can be the descendants of totipotent cells or obtained through induction of a non-pluripotent cell, such as an adult somatic cell, by forcing the expression of certain genes.

As used herein, the term “embryonic stem cells (ESCs),” also commonly abbreviated as ES cells, refers to cells that are pluripotent and derived from the inner cell mass of the blastocyst, an early-stage embryo. For purpose of the present invention, the term “ESCs” is used broadly sometimes to encompass the embryonic germ cells as well.

As used herein, the term “precursor cell” encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment.

In developmental biology, cellular differentiation is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

As used herein, the term “cellular constituents” are individual genes, proteins, mRNA expressing genes, and/or any other variable cellular component or protein activities such as the degree of protein modification (e.g., phosphorylation), for example, that is typically measured in biological experiments (e.g., by microarray, RNA sequencing, single cell RNA sequencing or immunohistochemistry) by those skilled in the art. Significant discoveries relating to the complex networks of biochemical processes underlying living systems, common human diseases, and gene discovery and structure determination can now be attributed to the application of cellular constituent abundance data as part of the research process. Cellular constituent abundance data can help to identify biomarkers, discriminate disease subtypes and identify mechanisms of toxicity.

As used herein, the term “organoid” is used to mean a 3-dimensional growth of mammalian cells in culture that retains characteristics of the tissue in vivo, e.g. prolonged tissue expansion with proliferation, multilineage differentiation, recapitulation of cellular and tissue ultrastructure, etc. Organoids can be obtained from iPSCs by guiding their differentiation from pluripotency into a specific tissue lineage using directed differentiation, or organoids can be obtained from tissue samples obtained from humans, and by isolating said tissue and culturing it in vitro.

### DETAILED DESCRIPTION

The human intestinal stem cell (ISC) niche supports ISC self-renewal and epithelial function, yet little is known about the development of the human ISC niche. Experiments described herein used single-cell mRNA sequencing (scRNA-seq) to interrogate the human intestine across 7-21 weeks post conception. Using these data coupled with marker validation in situ, molecular identities and spatial locations were assigned to several cell populations that comprise the ISC niche, and the cellular origins of many niche factors were determined. The source of WNT and RSPONDIN ligands closest to the stem cell niche were cells of the muscularis mucosa. EGF was predominantly expressed in the villus epithelium and the EGF-family member NEUREGULIN 1 (NRG1) was expressed by subepithelial cells identified as F3+/PDGFRAHI. Functional data from enteroid cultures showed that NRG1 and/or EREG improved cellular diversity, enhanced the stem cell gene signature, and performed equivalently to EGF enteroid forming assays, whereas EGF supported a secretory gene expression profile with less cellular diversity.

Accordingly, provided herein are methods and systems for differentiating, generating and/or maintaining intestinal organoids (e.g., enteroids) using NRG1 and/or EREG.

In some embodiments, intestinal organoids are generated from fetal intestinal tissue or tissue obtained from intestinal tissue after birth, ranging from childhood through adulthood.

In some embodiments, intestinal organoids are generated from stem cells. For example, in some embodiments, embodiments, intestinal organoids are generated from iPSC or other pluripotent stem cells, and are obtained using a process of directed differentiation.

Additional stem cells that can be used in embodiments in accordance with the present invention include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (UW-SCRMC); Novocell, Inc. (San Diego, Calif.); Cellartis AB (Goteborg, Sweden); ES Cell International Pte Ltd (Singapore); Technion at the Israel Institute of Technology (Haifa, Israel); and the Stem Cell Database hosted by Princeton University and the University of Pennsylvania. Indeed, embryonic stem cells that can be used in embodiments in accordance with the present invention include but are not limited to SA01 (SA001); SA02 (SA002); ES01 (HES-1); ES02 (HES-2); ES03 (HES-3); ES04 (HES-4); ES05 (HES-5); ES06 (HES-6); BG01 (BGN-01); BG02 (BGN-02); BG03 (BGN-03); TE03 (13); TE04 (14); TE06 (16); UC01 (HSF1); UC06 (HSF6); WA01 (H1); WA07 (H7); WA09 (H9); WA13 (H13); WA14 (H14).

In some embodiments, the stem cells are further modified to incorporate additional properties. Exemplary modified cell lines include but not limited to H1 OCT4-EGFP; H9 Cre-LoxP; H9 hNanog-pGZ; H9 hOct4-pGZ; H9 in GFPhES; and H9 Syn-GFP.

More details on embryonic stem cells can be found in, for example, Thomson et al., 1998, Science 282 (5391):1145-1147; Andrews et al., 2005, Biochem Soc Trans 33:1526-1530; Martin 1980, Science 209 (4458):768-776; Evans and Kaufman, 1981, Nature 292(5819): 154-156; Klimanskaya et al., 2005, Lancet 365 (9471): 1636-1641).

Alternative, pluripotent stem cells can be derived from embryonic germ cells (EGCs), which are the cells that give rise to the gametes of organisms that reproduce sexually. EGCs are derived from primordial germ cells found in the gonadal ridge of a late embryo, have many of the properties of embryonic stem cells. The primordial germ cells in an embryo develop into stem cells that in an adult generate the reproductive gametes (sperm or eggs). In mice and humans it is possible to grow embryonic germ cells in tissue culture under appropriate conditions. Both EGCs and ESCs are pluripotent. For purpose of the present invention, the term “ESCs” is used broadly sometimes to encompass EGCs.

In some embodiments, iPSCs are derived by transfection of certain stem cell-associated genes into non-pluripotent cells, such as adult fibroblasts. Transfection is typically achieved through viral vectors, such as retroviruses. Transfected genes include the master transcriptional regulators Oct-3/4 (Pouf51) and Sox2, although it is suggested that other genes enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include but are not limited to first generation iPSCs, second generation iPSCs in mice, and human induced pluripotent stem cells. In some embodiments, a retroviral system is used to transform human fibroblasts into pluripotent stem cells using four pivotal genes: Oct3/4, Sox2, Klf4, and c-Myc. In alternative embodiments, a lentiviral system is used to transform somatic cells with OCT4, SOX2, NANOG, and LIN28. Genes whose expression are induced in iPSCs include but are not limited to Oct-3/4 (e.g., Pou5fl); certain members of the Sox gene family (e.g., Sox1, Sox2, Sox3, and Sox15); certain members of the Klf family (e.g., Klf1, Klf2, Klf4, and Klf5), certain members of the Myc family (e.g., C-myc, L-myc, and N-myc), Nanog, and LIN28.

More details on induced pluripotent stem cells can be found in, for example, Kaji et al., 2009, Nature 458:771-775; Woltjen et al., 2009, Nature 458:766-770; Okita et al., 2008, Science 322(5903):949-953; Stadtfeld et al., 2008, Science 322(5903):945-949; and Zhou et al., 2009, Cell Stem Cell 4(5):381-384.

In some embodiments, examples of iPS cell lines include but not limited to iPS-DF19-9; iPS-DF19-9; iPS-DF4-3; iPS-DF6-9; iPS(Foreskin); iPS(IMR90); and iPS(IMR90).

The present invention, in some embodiments, provides methods for directing the differentiation of intestinal tissue or cells (e.g., stem cells) into human intestinal organoid tissue (e.g., enteroids) by culturing such tissues or cells in the presence of NRG1 and/or EREG.

In some embodiments, the culturing is in Matrigel. In some embodiments, the culturing is in other naturally or synthetically occurring extra cellular matrices or hydrogels. In some embodiments, the culture medium further comprises R-spondin 1, 2, or 3, and Noggin. In some embodiments, the culture medium does not comprise EGF. In some embodiments, the culture medium further comprises Epiregulin (EREG). In some embodiments, the culture medium further comprises Neuregulin 1 (NRG1).

In some embodiments, human intestinal organoids (e.g., enteroids) produced in vitro from the described methods can be used to screen drugs for intestinal tissue uptake and mechanisms of transport. For example, this can be done in a high throughput manner to screen for the most readily absorbed drugs, and can augment Phase 1 clinical trials that are done to study drug intestinal tissue uptake and intestinal tissue toxicity. This includes pericellular and intracellular transport mechanisms of small molecules, peptides, metabolites, salts.

In some embodiments, human intestinal organoids produced in vitro from the described methods can be used to identify the molecular basis of normal human intestinal development.

In some embodiments, human intestinal organoids produced in vitro from the described methods can be used to identify the molecular basis of congenital defects affecting human intestinal development.

In some embodiments, human intestinal organoids produced in vitro from the described methods can be used to correct intestinal related congenital defects caused by genetic mutations. In particular, mutation affecting human intestinal development can be corrected using iPSC technology and genetically normal human intestinal organoids produced in vitro from the described methods. In some embodiments, human intestinal organoids produced in vitro from the described methods can be used to generate replacement tissue.

In some embodiments, human intestinal organoids produced in vitro from the described methods can be used to generate replacement intestinal tissue for intestine related disorders.

In some embodiments, a diagnostic kit or package is developed to include human intestinal organoids produced in vitro from the described methods and based on one or more of the aforementioned utilities.

The invention provides a composition comprising a culture medium according to the invention and stem cells. The invention also provides a composition comprising a culture medium according to the invention and organoids. Furthermore, the invention provides a composition comprising a culture medium according to the invention. Furthermore, the invention provides a composition comprising a culture medium according to the invention and an extracellular matrix (e.g., Matrigel).

The invention also provides a composition comprising a culture medium of the invention, an extracellular matrix and human pluripotent stem cells. The invention also provides a composition comprising a culture medium of the invention, an extracellular matrix and human intestinal organoids.

The invention also provides a hermetically-sealed vessel containing a culture medium of the invention. Hermetically-sealed vessels may be preferred for transport or storage of the culture media or culture media supplements disclosed herein, to prevent contamination. The vessel may be any suitable vessel, such as a flask, a plate, a bottle, a jar, a vial or a bag.

The invention provides the use of human intestinal organoids or cells derived thereof in drug screening, (drug) target validation, (drug) target discovery, toxicology and toxicology screens, personalized medicine, regenerative medicine and/or as ex vivo cell/organ models, such as disease models.

Cells and human intestinal organoids cultured according to the media and methods of the invention are thought to faithfully represent the in vivo situation. This is true both for expanded populations of cells and organoids grown from normal tissue and for expanded populations of cells and organoids grown from diseased tissue. Therefore, as well as providing normal ex vivo cell/organ models, the organoids of the invention can be used as ex vivo disease models.

Organoids of the invention (e.g., human intestinal enteroids) can also be used for culturing of a pathogen and thus can be used as ex vivo infection models. Examples of pathogens that may be cultured using an organoid of the invention include viruses, bacteria, prions or fungi that cause disease in its animal host. Thus an organoid of the invention can be used as a disease model that represents an infected state. In some embodiments of the invention, the organoids can be used in vaccine development and/or production.

Diseases that can be studied by the organoids of the invention (e.g., human intestinal enteroids) thus include genetic diseases, metabolic diseases, pathogenic diseases, inflammatory diseases etc of the intestine and/or related to intestinal development.

The organoids of the invention (e.g., human intestinal enteroids) can be frozen and thawed and put into culture without losing their genetic integrity or phenotypic characteristics and without loss of proliferative capacity. Thus the organoids can be easily stored and transported. Thus in some embodiments, the invention provides a frozen organoid.

For these reasons the organoids or expanded populations of cells of the invention can be a tool for drug screening, target validation, target discovery, toxicology and toxicology screens and personalized medicine.

Accordingly, in a further aspect, the invention provides the use of an organoid or cell derived from said organoid according to the invention in a drug discovery screen, toxicity assay or in medicine, such as regenerative medicine. For example, the vascularized human intestinal organoid tissue having an intestine-specific EC transcriptional signature may be used in a drug discovery screen, toxicity assay or in medicine, such as regenerative medicine.

For preferably high-throughput purposes, said organoids of the invention (e.g., human intestinal enteroids) are cultured in multiwell plates such as, for example, 96 well plates or 384 well plates. Libraries of molecules are used to identify a molecule that affects said organoids. Preferred libraries comprise antibody fragment libraries, peptide phage display libraries, peptide libraries, lipid libraries, synthetic compound libraries or natural compound libraries. Furthermore, genetic libraries can be used that induce or repress the expression of one of more genes in the progeny of the stem cells. These genetic libraries comprise cDNA libraries, antisense libraries, and siRNA or other non-coding RNA libraries. The cells are preferably exposed to multiple concentrations of a test agent for a certain period of time. At the end of the exposure period, the cultures are evaluated. The term “affecting” is used to cover any change in a cell, including, but not limited to, a reduction in, or loss of, proliferation, a morphological change, and cell death.

In some embodiments, the organoids of the invention (e.g., human intestinal enteroids) can be used to test libraries of chemicals, antibodies, natural product (plant extracts), etc for suitability for use as drugs, cosmetics and/or preventative medicines.

The invention provides the use of human intestinal enteroids in regenerative medicine and/or transplantation. The invention also provides methods of treatment wherein the method comprises transplanting an organoid into an animal or human.

Human intestinal organoids are useful in regenerative medicine, for example in treatment of post-radiation and/or post-surgery repair of the intestinal epithelium, in the repair of the intestinal epithelium in patients suffering from inflammatory bowel disease such as Crohn's disease and ulcerative colitis, and in the repair of the intestinal epithelium in patients suffering from short bowel syndrome. Further use is present in the repair of the intestinal epithelium in patients with hereditary diseases of the small intestine/colon.

### Experimental

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

### EXAMPLE 1

### Methods

Isolating, establishing and maintaining human enteroids:

Fresh human fetal epithelium was isolated and maintained as previously described (Tsai et al., Cell. Mol. Gastroenterol. Hepatol. 1-12 2018). Once enteroids were established, healthy enteroids were manually selected under a stereoscope and bulk-passaged through a 30 G needle and embedded in Matrigel (Corning, 354234). For single-cell passaging, healthy enteroids were manually selected under a stereoscope and dissociated with TrypLE Express (Gibco, 12605-010) at 37° C. before filtering through 40 μm cell strainers. Cells were then counted using a hemocytometer (ThermoFisher) and embedded in Matrigel.

Media composition:

Culture media consisted of 25% LWRN conditioned media generated as previously described (Miyoshi and Stappenbeck, Nat. Protoc. 8, 2471-2482 2013; Tsai et al., 2018, surpa) and 75% Human 2× basal media (Advanced DMEM/F12 (Gibco, 12634-028); Glutamax 4 mM (Gibco, 35050-061); HEPES 20 mM (Gibco, 15630-080); N2 Supplement (2×) (Gibco, 17502-048), B27 Supplement (2×) (17504-044), Penicillin-Streptomycin (2×) (Gibco, 15140-122), N-acetylcysteine (2 mM) (Sigma, A9165-25G), Nicotinamide (20 mM) (Sigma, N0636-061)). This culture media was the base media for the eight culture conditions with varied concentrations of rhEGF (R&D, 236-EG) and rhNRG1 (R&D, 5898-NR-050) as follows: 100 ng/mL EGF with 0, 1, 10, and 100 ng/mL NRG1; 100 ng/mL NRG1 with 0, 1, and 10 ng/mL EGF; and culture media with neither EGF nor NRG1.

Human subjects:

Normal, de-identified human fetal intestinal tissue was obtained from the University of Washington Laboratory of Developmental Biology. All human tissue used in this work was deidentified and was conducted with approval from the University of Michigan IRB.

Experimental design of enteroid cultures:

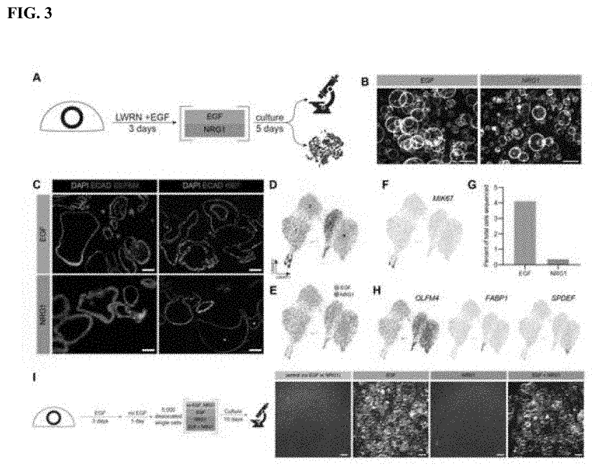

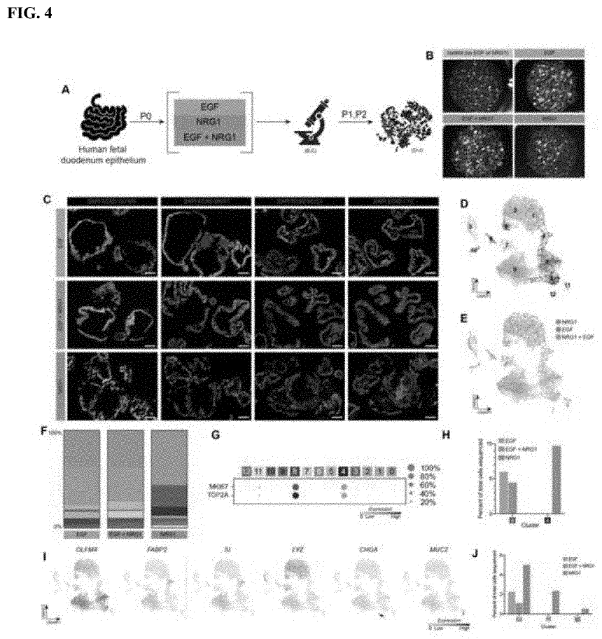

The enteroid experiments in FIGS. 3 and 4 were carefully conducted to reduce batch effects in scRNA-seq data. All experiments comparing different treatment groups (EGF, NRG1, etc) were carried out in parallel, with experiments and treatments being conducted at the same time. Cells were harvested and dissociated into single cell suspensions in parallel (see below). Since the 10× Chromium system allows parallel processing of multiple samples at a time, cells were captured (Gel bead-in-Emulsion—GEMS) and processed (library prep) in parallel. All samples were sequenced across the same lane(s) on a Novaseq 6000.

Single cell dissociation:

To dissociate human fetal tissue to single cells, fetal duodenum was first dissected using forceps and a scalpel in a petri dish filled with ice-cold 1× HBSS (with Mg²⁺, Ca²⁺). Whole thickness intestine was cut into small pieces and transferred to a 15 mL conical tube with 1% BSA in HBSS. Dissociation enzymes and reagents from the Neural Tissue Dissociation Kit (Miltenyi, 130-092-628) were used, and all incubation steps were carried out in a refrigerated centrifuge pre-chilled to 10° C. unless otherwise stated. All tubes and pipette tips used to handle cell suspensions were pre-washed with 1% BSA in 1× HBSS to prevent adhesion of cells to the plastic. Tissue was treated for 15 minutes at 10° C. with Mix 1 and then incubated for 10 minute increments at 10° C. with Mix 2 interrupted by agitation by pipetting with a P1000 pipette until fully dissociated. Cells were filtered through a 70 μm filter coated with 1% BSA in 1× HBSS, spun down at 500 g for 5 minutes at 10° C. and resuspended in 500 μl 1× HBSS (with Mg²⁺, Ca²⁺). 1 mL Red Blood Cell Lysis buffer was then added to the tube and the cell mixture was placed on a rocker for 15 minutes in the cold room (4° C.). Cells were spun down (500 g for 5 minutes at 10° C.), and washed twice by suspension in 2 mL of HBSS +1% BSA, followed by centrifugation. Cells were counted using a hemocytometer, then spun down and resuspended to reach a concentration of 1000 cells/μL and kept on ice. Single cell libraries were immediately prepared on the 10× Chromium at the University of Michigan Sequencing Core facility with a target of 5000 cells. The same protocol was used for single cell dissociation of healthy enteroids manually collected under a stereoscope. A full, detailed protocol of tissue dissociation for single cell RNA sequencing can be found at www.jasonspencelab.com/protocols.

Single cell library preparation and transcriptome alignment:

All single-cell RNA-seq sample libraries were prepared with the 10× Chromium Controller using either the v2 or v3 chemistry. Sequencing was performed on an Illumina HiSeq 4000 or NovaSeq 6000 with targeted depth of 100,000 reads per cell. Default alignment parameters were used to align reads to the pre-prepared hg19 human reference genome provided by the 10× Cellranger pipeline. Initial cell demultiplexing and gene quantification were also performed with the default 10× Cellranger pipeline.

Primary tissue collection, fixation and paraffin processing:

Human fetal intestine tissue samples were collected as ˜0.5 cm fragments and fixed for 24 hours at room temperature in 10% Neutral Buffered Formalin (NBF), and washed with UltraPure Distilled Water (Invitrogen, 10977-015) for 3 changes for a total of 2 hours. Tissue was dehydrated by an alcohol series diluted in UltraPure Distilled Water (Invitrogen, 10977-015). Tissue was incubated for 60 minutes each solution: 25% Methanol, 50% Methanol, 75% Methanol, 100% Methanol. Tissue was stored long-term in 100% Methanol at 4° C. Prior to paraffin embedding, tissue was equilibrated in 100% Ethanol for an hour, and then 70% Ethanol. Tissue was processed into paraffin blocks in an automated tissue processor (Leica ASP300) with 1 hour changes overnight.

Enteroid collection, fixation and paraffin processing:

Enteroids were allowed to grow in Matrigel for several days following passaging. Once established, Enteroids in Matrigel were transferred gently with a cut pipette P1000 tip into a petri dish filled with cold DMEM/F12. Enteroids are then manually dissected from Matrigel under a dissecting stereomicroscope using fine forceps and transferred to a microcentrifuge tube. Enteroids are left upright for several minutes until they gravity sediment to at the bottom of the tube, at which time as much media as possible is gently withdrawn. HISTOGEL (Thermo scientific, HG-4000-012) is slowly added to cover the enteroids following the manufacturers protocol. Once HISTOGEL has solidified, Histogel-embedded enteroids are transferred to a 5 mL conical tube and fixed in 10% NBF overnight at room temperature. Once fixed, they are processed into paraffin as described above, sectioned and staining for FISH and IF described below.

Multiplex Fluorescent In Situ Hybridization (FISH) and immunofluorescence (IF):

Paraffin blocks were sectioned to generate 5 μm-thick sections within a week prior to performing in situ hybridization. All materials, including the microtome and blade, were sprayed with RNase-away solution prior to use. Slides were baked for 1 hour in a 60° C. dry oven the night before, and stored overnight at room temperature in a slide box with a silicone desiccator packet, and with seams sealed using parafilm. The in situ hybridization protocol was performed according to the manufacturer's instructions (ACD; RNAscope multiplex fluorescent manual protocol, 323100-USM) under standard antigen retrieval conditions and 30 minute protease treatment. Immediately following the HRP blocking for the C2 channel of the FISH, slides were washed three times for 5 minutes in PBS, then transferred to blocking solution (5% Normal Donkey Serum in PBS with 0.1% Tween-20) for 1 hour at room temperature. Slides were then incubated in primary antibodies overnight at 4° C. in a humidity chamber. The following day, excess primary antibodies were rinsed off through a series of PBS washes. Secondary antibodies and DAPI (1 μg/ml) were added and slides were incubated at room temperature for 1 hour. Excess secondary antibodies were rinsed off through a series of PBS washes, and slides were mounted in ProLong Gold (TermoFisher, P36930). All imaging was done using a NIKON A1 confocal and images were assembled using Photoshop CC. Z-stack series were captured and compiled into maximum intensity projections using NIS-Elements (Nikon). Imaging parameters were kept consistent for all images within the same experiment and any post-imaging manipulations were performed equally on all images from a single experiment.

Single-cell in silico analysis:

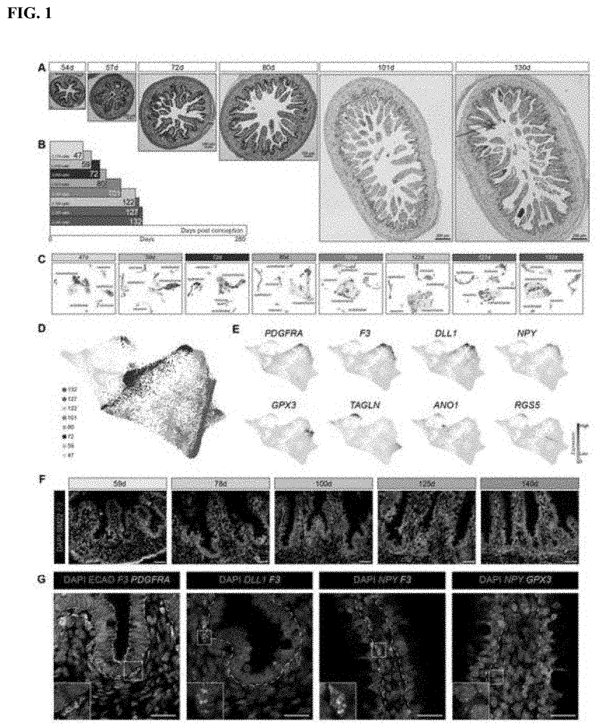

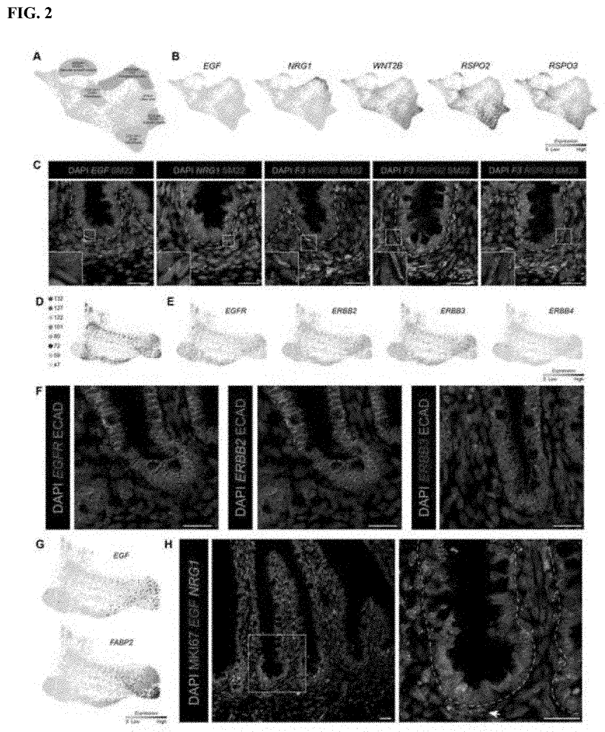

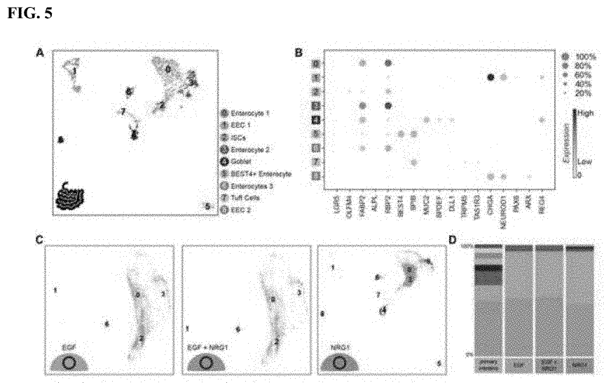

All in silico analyses downstream of gene quantification were done using Scanpy with the 10× Cell Ranger derived gene by cell matrices (Wolf et al., Genome Biol. 19, 15 2018). For primary human tissue sample analysis in FIGS. 1 and 2, all samples were filtered to remove cells with less than 1000 or greater than 9000 genes, less than 3500 or greater than 25000 unique molecular identifier (UMI) counts per cell. Ambient/background signal was removed from the data using CellBender. “Remove-background” was used at 200 epochs to remove ambient RNA counts from all fetal intestine samples, and the de-noised data matrix was used for subsequent analysis (Fleming et al., BioRxiv 791699 2019). De-noised data matrix read counts per gene were log normalized prior to analysis. After log normalization, 2000-3000 highly variable genes were identified and extracted. The normalized expression levels then underwent linear regression to remove effects of total reads per cell and cell cycle genes, followed by a z-transformation. Dimension reduction was performed using principal component analysis (PCA) and then uniform manifold approximation and projection (UMAP) on the top 9 principal components (PCs) and 30 nearest neighbors for visualization on 2 dimensions (McInnes et al., J. Open Source Softw. 3, 861 2018; Polański et al., Bioinformatics 2019). Clusters of cells within the data were calculated using the Louvain algorithm within Scanpy with a resolution of 0.6. For FIGS. 1 and 2, combined time series data for mesenchymal and epithelial cells were integrated using Harmony to generate augmented affinity matrices and plotted as force-directed layouts with ForceAtlas2 (Jacomy et al., PLoS One 9, e986792014; Nowotschin et al., Nature 569, 361-367 2019). For FIGS. 3, 4 and 5, all samples were filtered to remove cells with too few or too many genes (FIG. 3—<2000, >9000; FIG. 4 <250, >8000; FIG. 5 <500, >3000) or with high unique molecular identifier (UMI) counts per cell (FIG. 3—100000; FIG. 4—10000; FIG. 5—10000), and a fraction of mitochondrial genes greater than 0.1-0.25. Data matrix read counts per gene were log normalized prior to analysis. After log normalization, 2000-3000 highly variable genes were identified and extracted. For FIG. 3, the normalized expression levels then underwent linear regression to remove effects of total reads per cell and mitochondrial transcript fraction. Data was then scaled by z-transformation. Dimension reduction was performed using principal component analysis (PCA) and then uniform manifold approximation and projection (UMAP) on the top 11-20 principal components (PCs) and 15-30 nearest neighbors for visualization on 2 dimensions (McInnes et al., J. Open Source Softw. 3, 861 2018; Polański et al., 2019, supra).

Clusters of cells within the data were calculated using the Louvain algorithm within Scanpy with a resolution of 0.2-0.4. Following initial PCA dimension reduction and UMAP visualization, further de-noising was not carried out for this analysis given the distinct cell clusters. Scanpy's Ingest functionality was used to map enteroids onto primary human fetal epithelial cells. Epithelial cells were identified and extracted from a data matrix to include intestinal epithelial cells from ages 101, 122, 127, and 132 days (ArrayExpress: E-MTAB-9489). Epithelial cells were annotated using canonical genes. The extracted epithelial cell matrix then again underwent log normalization, variable gene extraction, z transformation and dimension reduction to obtain a reference embedding. Ingest was then run to project each of the individual enteroid datasets onto the epithelial reference map.

### EXAMPLE 2

### Interrogating the Developing Human Small Intestine with Single Cell Resolution

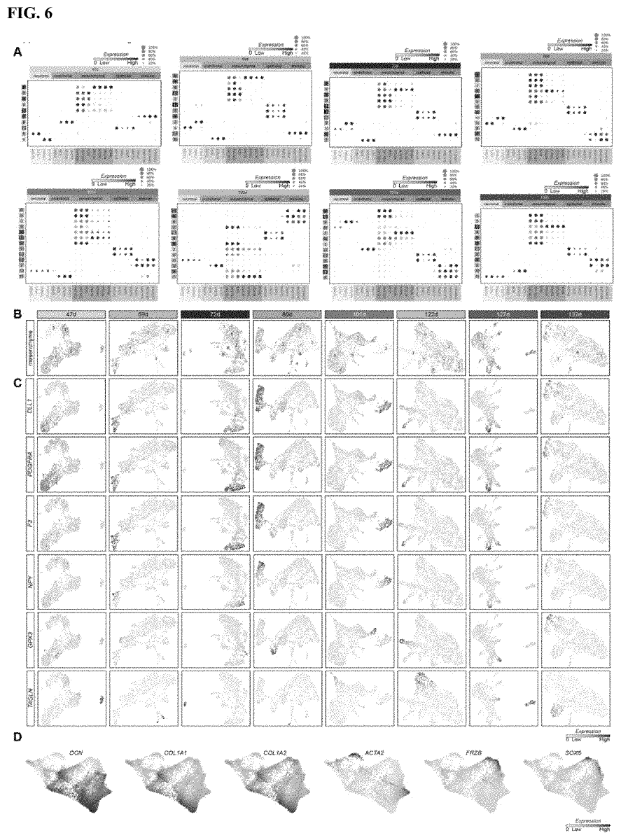

Given that little is known about mesenchymal cell heterogeneity within the fetal human intestine, experiments were conducted to better understand the mesenchymal cell populations that make up the developing human ISC niche. To do this, samples of human fetal duodenum starting just after the onset of villus morphogenesis (47 days post conception; 47 d) with samples interspersed up to the midpoint (132 d) of typical full-term (280 d) and performed histological and molecular analysis (FIG. 1A-B) were used. Major physical changes occur throughout this developmental window with rapid growth in length and girth, along with the formation of villi and crypt domains within the epithelium and increased organization and differentiation of smooth muscle layers within the mesenchyme (Chin et al., Cell Dev. Biol 2017) (FIG. 1A). In order to capture the full complement of cell types that contribute to the developing human intestine, full thickness intestinal tissue was dissociated from 8 duodenal specimens ranging between 47 d-132 days post conception and used for scRNA-seq experiments to sequence 2,830-3,197 cells per specimen after filtering and ambient RNA removal. 24,783 total cells were used in the analysis after passing computational quality filtering (FIG. 1B) Following dimensional reduction and visualization with UMAP (Becht et al., Nat. Biotechnol. 37, 38-47 2019; Wolf et al., 2018, supra), canonical genes were used to annotate each sample individually by identifying major cell classes including epithelial, mesenchymal, endothelial, enteric nervous and immune cells (FIG. 1C, FIG. 6A). In order to focus the analysis on the mesenchymal niche populations found in each sample, the mesenchyme was computationally extracted and re-clustered, and a population of PDGFRA^{HI} cells, which have also been described in mice (McCarthy et al., Cell Stem Cell 26, 391-402.e5 2020), and ACTA2+/TAGLN+ 120 smooth muscle cells (FIG. 1E, FIG. 6C) were annotated. Additional sub-clusters and gene expression patterns not previously described in mice were identified (FIG. 6B). Given the dramatic morphological changes that take place across this development time (FIG. 1A), Harmony (Nowotschin et al., Nature 569, 361-367 2019), an algorithm that allows interrogation of scRNA-seq data across discrete time points, was used (FIG. 1D-E).

Force directed layouts following Harmony implementation ordered cells broadly according to their developmental age (days post conception) (FIG. 1D). The 47 d cells were largely separate from other time points with the exception of an ACTA2^{HI}/TAGLN^{HI}/RGS5⁺ population of vascular smooth muscle cells (VSMC) (Muhl et al., Nat. Commun. 11, 3953 2020) (FIG. 1D-E). Cells were then ordered according to developmental time, with cells from samples older than 101 d (101 d, 122 d, 127 d, 132 d) clustering together. In addition to the VSMC population, this analysis supported the emergence of several mesenchymal populations, including a PDGFRA^{HI}/F3^{HI} population, a GPX3HI population, a TAGLN^{HI}/RGS5⁻ smooth muscle population and a prominent clusters of cells defined as fibroblasts based on their expression COLLAGEN genes (COL1A1, COL1A2) and DECORIN (DCN) (FIG. 1E, FIG. 6D) (Kinchen et al., Cell 175, 372-3862018). F3 was recently shown to be expressed in a population of mesenchymal cells that is adjacent to the human colonic epithelium (Kinchen et al., 2018, supra) and identified that these cells were additionally characterized by their enrichment of NPY, DIL1, FRZB and SOX6 (FIG. 1E, FIG. 6B-D).

### EXAMPLE 3

### Mesenchymal Cell Lineages Emerge Across Developmental Time

Force directed layouts following Harmony implementation indicated that different mesenchymal cell populations emerge across developmental time. For example, F3^{HI}/PDGFRA^{HI} cells emerged after approximately 59 days, while PX3^{HI}/F3^{LO}/PDGFRA^{LO} and TAGLN^{HI}/RGS5⁻ smooth muscle cells emerge after approximately 80 days. To corroborate scRNA-seq analysis, a combinatorial staining approach, utilizing multiplexed fluorescent in situ hybridization (FISH) and immunofluorescence (IF) was used to examine F3 mRNA and SM22, the protein product of the TAGLN gene (FIG. 1F). It was found that F3 was not expressed at 59 d but was clearly expressed in the villus mesenchyme by 78 d, with expression becoming more restricted to the subepithelial cells as time progressed. It was observed that F3 was expressed in the scRNA-seq at 59 days, however, it is possible that since fetal tissue staging is an approximation, samples may be slightly older or younger than their actual labeling, explaining slight discrepancies such as this. SM22 was expressed in the 59 d intestine, but only in the outermost muscularis externa layer. SM22 expression in the muscularis mucosa, the layer closest to the intestinal epithelium and adjacent to the proliferative crypt domains, was first observed as poorly organized cells near the epithelium at 100 d that became more organized after this time point. Single cell analysis and FISH/IF collectively indicate that the mesenchyme in the early fetal intestine is naïve and that mesenchymal cell emergence coincides with the formation of proliferative intervillus/crypt domains.

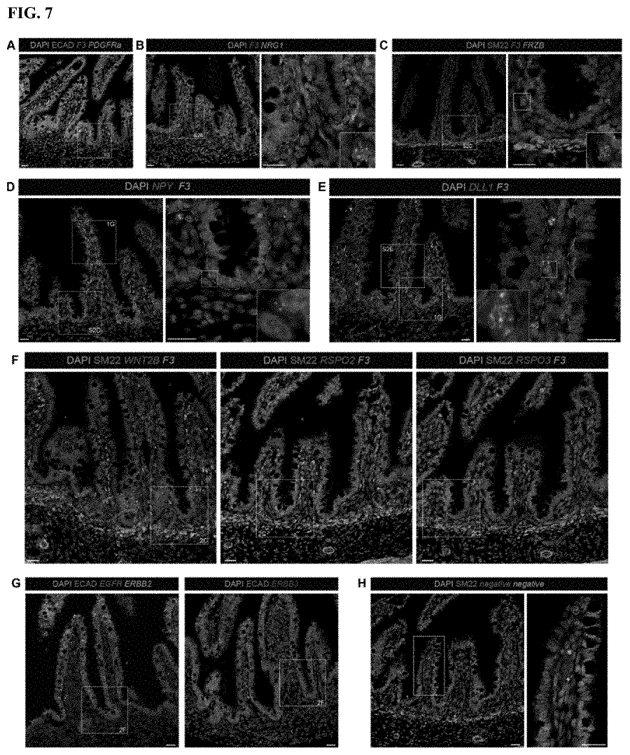

To understand how mesenchymal cell populations are spatially organized within the tissue after 100 days, FISH/IF was used to observe that F3^{HI} cells co-express PDGFRA, DLL1 and NPY (FIG. 1E). F3^{HI}/NPY^{HI} cells were restricted to the subepithelial cells lining the villus (FIG. 1E), but not that of the crypt (FIG. 7D). GPX3^{HI}/F3^{LO}/PDGFRA^{LO} cells were most abundant within the core of intestinal villi and are observed sitting adjacent to NPYHI cells (FIG. 1E).

### EXAMPLE 4

### Identifying Putative Human ISC Niche Factors in the Developing Gut

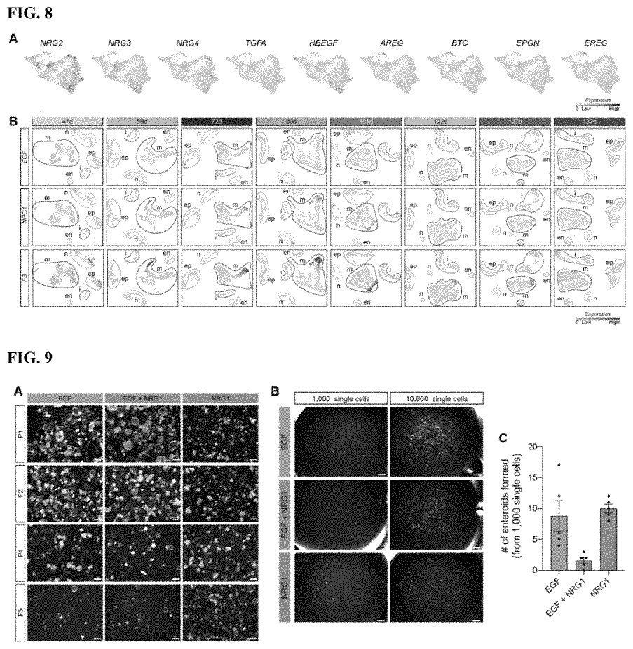

It has been demonstrated that several niche factors allow adult and developing human and murine intestinal epithelium to be cultured ex vivo as organoids (Capeling et al., Stem Cell Reports 12, 381-394 2019; Finkbeiner et al., Stem Cell Reports 4, 1140-1155 2015; Fordham et al., Cell Stem 2013; Hill et al., Elife 6, e29132 2017; Kraiczy et al., Gut gutjnl-2017-314817-14 2017; Sato et al., 2009 Nature 459, 262-265, Gastroenterology 141, 1762-1772 2011). These factors often include WNT and RSPO ligands, BMP/TGFβ antagonists and EGF, and are based on defined growth conditions that allow expansion of intestinal epithelium in vitro (Sato et al., 2009, 2011, supra). Efforts have been made to determine more physiological niche factors for in vitro culture systems based on observed in vivo niche cues (Fujii et al., Cell Stem Cell 23, 787-793.e6 2018), however niche factors have not been interrogated in the developing human gut using high resolution technologies such as scRNA-seq. To identify putative niche factors, it was first determined which cells within the human fetal intestine expressed known niche factors. It was observed that F3^{HI}/PDGFRA^{HI} subepithelial cells, and GPX3^{HI}/F3^{LO}/PDGFA^{LO} villus core cells lacked robust expression of most known niche factors (FIG. 2A-B), whereas the WNT pathway members with the highest expression were RSPO2, RSPO3 and WNT2B, which are expressed in the TAGLN^{HI}/RGS5⁻ smooth muscle cells and COL1A1^{HI} fibroblast population, but are not expressed by the F3^{HI}/PDGFRA^{HI} subepithelial cells (FIG. 2A-B). EGF is a critical driver of proliferation in murine enteroid culture (Basak et al., Cell Stem Cell 20, 177-190.e4 2017); however, EGF expression was not observed in the mesenchyme, whereas the EGF family member NRG1 was abundant in the F3^{HI}/PDGFRA^{HI} cell population (FIG. 2A-B). Of note, NRG1 was the most robust EGF family member expressed in the F3^{HI}/PDGFRA^{HI} cell population (FIG. 8A). IF for SM22, combined with FISH for RSPO2, RSPO3, WNT2B, EGF and NRG1 revealed expression patterns that were consistent with scRNA-seq data (FIG. 2C, FIG. 7F).

Given the importance of EGF for in vitro enteroid culture, it was further interrogated whether EGF and EGF receptors are expressed in the developing intestinal epithelium via scRNA-seq and FISH. All epithelial cells were extracted and re-clustered, and the data was visualized using a force directed layout following application of Harmony (FIG. 2D). The ERBB receptors, including EGFR, ERBB2 and ERBB3 were broadly expressed throughout the epithelium, a finding that was confirmed by FISH, while ERBB4 was not expressed (FIG. 2E-F). While EGF is not expressed in the intestinal mesenchyme (FIG. 2A-C), it was observed that EGF is expressed in a small subset of differentiated epithelial FABP2^{HI} enterocytes (FIG. 2G), a finding that was supported using co-FISH/IF and showed EGF expression is low/absent from the proliferative crypt domain, but is expressed several cell diameters above the MKI67+ crypt region and throughout the villus epithelium (FIG. 2D). On the other hand NRG1 is expressed in F3^{HI}/PDGFRA^{HI} subepithelial cells adjacent to the crypt (FIG. 2C,H; FIG. 7B).

### EXAMPLE 5

### NRG1 Does Not Support Proliferation and Growth in Established Enteroid Cultures

Based on the expression pattern of NRG1, it was hypothesized that it may act as an ERBB niche signaling cue and may be physiologically relevant in vitro based on its localization and proximity to ISCs within the developing intestine in vivo. To interrogate the effects of NRG1 and EGF on the intestinal epithelium, established human fetal duodenum derived epithelium-only intestinal enteroids (established from a 142 d specimen) were split in culture using standard growth conditions (WNT3A/RSPO3/NOG plus EGF) into two groups. One group of enteroids was cultured in standard media with EGF (100 ng/mL), the other was grown without EGF and was instead supplemented with NRG1 (100 ng/mL) (FIG. 3A). Following growth for 5 days in EGF or NRG1, enteroids did not appear phenotypically different (FIG. 3B). Upon interrogation using immunofluorescence, it was observed that EGF-grown cultures had both OLFM4+ and OLFM4− enteroids and that enteroids in this group were highly proliferative based on KI67 staining (FIG. 3C). NRG1 treated enteroids appeared to have more uniform OLFM4 expression but also had fewer KI67+ cells per field of view. To more closely interrogate these differences, each group was subjected to scRNA-seq to investigate transcriptional differences.

To reduce any chances of batch effect, all processing for single cell sequencing for these groups was carried out at the same time in parallel, libraries were prepared in parallel, and samples were sequenced on the same lane. Despite varying only EGF or NRG1 in the culture, a difference in gene expression was observed between the two groups as visualized in UMAP plots illustrated by near complete independent clustering of cells by culture media composition (FIG. 3D-E). The exception to this was cluster 4, which expressed proliferation markers (MKI67, TOP2A), and had a contribution from both samples (FIG. 3E-F). Cluster 4 appeared to have a higher number of cells from the EGF grown enteroids, and proportionally ˜4% (115/2,789) of cells from the EGF treated enteroids were in this cluster whereas <0.5% (10/2,720) of cells from the NRG1 treated enteroids were in this cluster (FIG. 3G). These data support the MKI67 immunofluorescence staining indicating that NRG1 had reduced proliferation. Enteroids from both groups broadly expressed OLFM4, although it appeared that expression levels were slightly higher when grown in NRG1, indicating that these samples generally had little heterogeneity and were largely comprised of undifferentiated stem cells (FIG. 3H). Consistent with this notion, differentially expressed genes associated with each cluster did not include genes canonically associated with differentiated cell types. A subset of cells in the NRG1 clusters expressed FAPB1 (expressed in enterocytes (Guilmeau et al., Histochem. Cell Biol. 128, 115 2007)) and a subset expressed SPDEF (expressed in secretory progenitors (Gregorieff et al., Gastroenterology 137, 1333 2009; Noah et al., Cell Res. 316, 452-465 2010)) (FIG. 3I). These data indicate that some cells in the NRG1 cultures may be in the early stages of differentiation; however, expression patterns were not sufficiently different to form distinct clusters.

To functionally evaluate the observation that proliferation was reduced in NRG1 treated enteroids, enteroids were bulk passaged and allowed to expand for 3 days in standard (EGF 100 ng/mL) growth conditions. EGF was removed for 24 hours, enteroids were dissociated into a single-cell suspension and plated 5,000 cells per droplet of Matrigel. Immediately upon seeding single cells, standard growth media supplemented with no-EGF (control), with EGF (100 ng/mL) only, with NRG1 (100 ng/mL) only, or with NRG1 (100 ng/mL) and EGF (100 ng/mL) was added (FIG. 3J). Robust re-establishment of enteroids was observed after 10 days in the standard EGF condition. In contrast, almost no enteroid recovery was observed in the control and in the NRG1-only supplemented cultures, whereas this growth defect was rescued in the NRG1 plus EGF condition (FIG. 3J). These functional results further support that EGF supports enhanced proliferation relative to NRG1 in established enteroid cultures.

### EXAMPLE 6

### Long-Term Enteroid Growth in NRG1 is Associated with Increased Epithelial Diversity In Vitro

The previous experiment was conducted with enteroids that had been established and expanded in long-term culture with EGF, and the experimental data (FIG. 3) supported that these cultures were highly dependent on EGF for proliferation. To determine the effects of different EGF-family members on establishment and long-term growth of enteroids, freshly isolated intestinal crypts were cultured in LWRN media supplemented with no EGF/NRG1 (control) EGF (100 ng/mL), NRG1 (100 ng/mL) or a combination of EGF and NRG1 (100 ng/mL each) (FIG. 4A). These cultures were used to carry out long-term passaging, imaging, quantitative enteroid forming assays and scRNA-seq (FIG. 9). Enteroids were successfully established in all conditions (FIG. 4B). All conditions successfully underwent serial passaging, with the exception of the controls (no EGF/NRG1), which failed to expand beyond initial plating (Passage 0; P0). To determine the effects of different growth conditions on enteroid forming ability, a quantitative single cell passaging assay was performed on surviving cultures at P2 (FIG. 9B-C). To do this, the three treatment groups were dissociated into single cells and plated 1,000 single cells (FIG. 9B) per droplet of Matrigel, allowed to grow for 11 days, and the number of recovered enteroids were quantitated (FIG. 9C). All groups re-established enteroids, albeit at a low efficiency, and there was no difference in enteroid forming efficiency between the EGF and NRG1 groups.

Examining all three groups that remained after passaging (EGF, NRG1, EGF/NRG1) by FISH or immunofluorescence revealed that OLFM4 was expressed in all conditions, and MKI67 did not appear different per field of view (FIG. 4C). The NRG1-only group appeared to have more MUC2 staining within the enteroid lumen, whereas both groups that included EGF (EGF-only, NRG1/EGF) had widespread LYZ expression within the epithelial cells (FIG. 4C). Given the different IF staining patterns of MUC2 and LYZ observed when comparing treatment groups (FIG. 4E), the cellular makeup and molecular signatures of these enteroids was investigated using scRNA-seq. For each group 3,262 cells grown in 100 ng/mL EGF, 7,350 cells grown in 100 ng/mL NRG1 and 2,593 grown in 100 ng/mL NRG1/EGF ere sequenced. UMAP dimensional reduction showed that the NRG1 treated enteroids clustered distinctly from the EGF-only enteroids, and indicated that the NRG1/EGF enteroids shared a high degree of molecular similarity with EGF-only enteroids since these samples overlaped in the clustering (FIG. 4E-F). Examining the cluster distribution for each sample, it was evident that EGF and EGF/NRG1 enteroids both contributed to the same clusters (clusters 1, 2, 8, 7), while NRG1 contributed to many distinct clusters (0, 3, 4, 11, 12) (FIG. 4F). Upon interrogation of genes associated with various clusters, proliferation genes were associated with two clusters—Cluster 4 (NRG1) and Cluster 8 (EGF and EGF/NRG1) (FIG. 4G-H). Several clusters expressed the stem cell marker OLFM4 (EGF, EGF/NRG1—clusters 1, 2, 8; NRG1—clusters 0, 3, 4). Cluster 6 had a contribution from all 3 groups and expressed enterocyte genes (SI, DPP4, FAPB2). Unique to the NRG1 grown enteroids, cluster 11 expressed genes associated with secretory progenitor cells and goblet cells (SPDEF and MUC2), and cluster 12 expressed genes associated with Enteroendocrine cells (CHGA) (FIG. 4I). LYZ expression was also investigated given the immunofluorescence staining results. LYZ was expressed at higher levels in the EGF and NRG1/EGF enteroids, as indicated by IF; however, low level expression was also observed broadly in the NRG1 treatment group (FIG. 4I). LYZ is canonically associated with Paneth cells; however, the fetal intestine does not possess Paneth cells until after 21 weeks post conception (Elmentaite et al., BioRxiv 2020.02.06.937110 2020; Finkbeiner et al., 2015, supra). Given that the enteroids used here were generated from specimens earlier than 21 weeks (replicate experiments utilized 105 d, 135 d specimens), it is unlikely that LYZ expression is associated with Paneth cells. Taken together, this data shows that both EGF and NRG1 can promote long term survival of freshly established enteroids, but that they have a differing impact on gene expression and cellular diversity.

### EXAMPLE 7

### Mapping to an Intestinal Reference Reveals Cellular Heterogeneity in Enteroids

In order to further interrogate cellular heterogeneity in enteroids grown in EGF, NRG1 and EGF/NRG1, the human fetal epithelium was used as a high-dimensional search space to determine the potential correspondence enteroids and their in vivo counterparts. To do this, the Ingest function (Wolf et al., Genome Biol. 19, 15 2018), which uses an annotated reference dataset that captures the biological variability of interest, and projects new data onto the reference was used. The major epithelial cell populations in the human fetal intestine were defined using the four samples that were older than 100 days (101-132 d). These samples were chosen based on the force directed layouts following Harmony augmentation, which supported that major changes in development/differentiation were not taking place across these times (FIG. 1-2). Eight epithelial cell types were defined based on published data, including intestinal stem cells (ISCs, cluster 2—LGR5, OLFM4), enterocytes (cluster 0 and 3—FABP2, ALPI, RBP2) (Haber et al., Nat. Publ. Gr. 551, 333-339 2017), BEST4+ enterocytes (cluster 5—BEST4, SPIB) (Elmentaite et al., 2020, supra), goblet cells and goblet cell precursors (cluster 4—MUC2, SPDEF, DLL1) (Okamoto et al., Liver Physiol. 296, G23-G35 2008), tuft cells (cluster 7—TRPM5, TAS1R3, SPIB) (Van Es et al., Proc. Natl. Acad. Sci. U.S.A. 116, 26599-26605 2019; Howitt et al., ImmunoHorizons 4, 23-32 2020; Kaske et al., BMC Neurosci. 8, 49 2007), enteroendocrine cells (EECs, clusters 1 and 8—CHGA, NEUROD1, PAX6, ARX, REG4) (Beucher et al., PLoS One 7, e36449 2012; Du et al., Dev. Biol. 2012; Gehart et al., Cell 176 2019; Haber et al., Nat. Publ. Gr. 551, 333-339 2017) (FIG. 5A, B). Ingest was used to map enteroids grown in EGF, EGF/NRG1 or NRG1 onto the in vivo epithelium (FIG. 5C), and determine the proportion of cells that mapped to each in vivo cell type, which was compared with the distribution of cells seen in the primary intestine (FIG. 5D).

These results supported the observations made in FIG. 4. EGF and EGF/NRG1 samples shared similar distribution patterns, with the majority of cells from both conditions mapping to ISCs and enterocytes, with a minor population mapping to EECs (FIG. 5C-D). NRG1 treated enteroids mapped to all cell types, including goblet cells (cluster 4), GHRL+ EECs (cluster 8), tuft cells (cluster 7), BEST4+ enterocytes (cluster 5), which were not present in EGF or EGF/NRG1 grown enteroids in this analysis (FIG. 5C-D). These results further support that NRG1 grown enteroids have enhanced cellular differentiation relative to enteroids grown in EGF.

### EXAMPLE 8

### EREG Contributions to ISC Niche

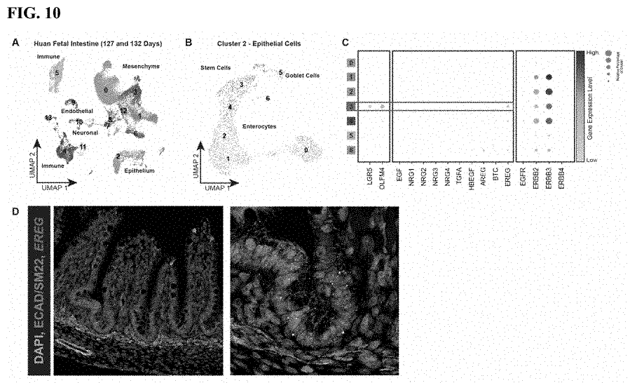

To interrogate additional factors that contribute to the developing human intestinal stem cell (ISC) niche, the human fetal intestine was surveyed at 127 and 132 days post conception using single cell RNA sequencing (scRNA-seq) technology. These time points mark approximately halfway through the 280-day gestation period and occur after most major developmental events in the human intestine. Full thickness intestinal tissue from both time points were dissociated to a single cell suspension and sent for single cell RNA sequencing. A total of 22,132 cells were sequenced between all samples, and after initial prefiltering, 19,748 cells were used for further computational analysis. Following dimensional reduction and visualization with UMAP, common canonically expressed genes were used to annotate the identity of major cell type clusters (FIG. 10A). One epithelial cluster, six mesenchymal clusters, an endothelial cluster, a neural cluster, and four immune clusters were identified. To focus specifically on the ISC niche, the epithelial cluster, cluster 2, was computational extracted and re-clustered and the major cell types of the intestinal epithelium were annotated using canonically expressed markers (FIG. 10B). To investigate EGF family ligand members' role in the niche, expression of all the family members were assayed. Cluster 3 was annotated as the ISCs due to robust LGR5 and OLFM4 expression and of the EGF family ligands, Epiregulin (EREG) was most widely enriched in this cluster indicating a potential role in the niche (FIG. 10C). To spatially confirm EREG's location in vivo, fluorescent in situ hybridization (FISH) on matched 127 day old fetal tissue was used. EREG expression was enriched in the crypts (FIG. 10D). These findings provided evidence that EREG may play a role in the ISC niche based on its close proximity to the ISCs.

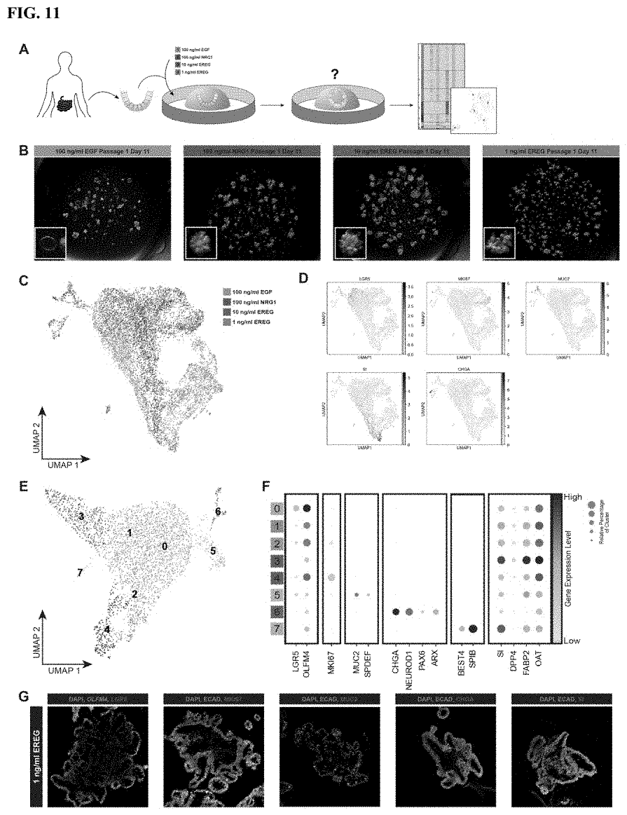

To elucidate EREG's role in the ISC niche, intestinal epithelial only organoids, herein referred to as enteroids, were established from fetal duodenal samples in EGF (control), NRG1 (Holloway et al.), and varying conditions of EREG (FIG. 11A). Morphological differences between culture conditions were immediately evident with low concentrations of EREG forming a budding morphology as compared to the standard cystic morphology observed in EGF grown enteroids (FIG. 11B). scRNA-seq analysis of these four culture conditions revealed EREG and NRG1 grown enteroids clustering away from a distinct EGF only population and featured more differentiated cell types not seen in EGF grown cultures (FIGS. 11C and 11D). The lowest concentration of EREG, 1 ng/ml EREG in 25% LWRN media, produced the most differentiated cell types including stem cells (markers: LGR5, OLFM4), goblet cells (markers: MUC2, SPDEF), enteroendocrine cells (markers: CHGA, NEUROD1, PAX6, ARX), BEST4+ Enterocytes (markers: BEST4, SPIB), and multiple enterocyte populations (markers: SI, DPP4, FABP2, OAT) (FIGS. 11E and 2F). Spatial characterization of these cell populations revealed crypt like domains with stem cells and proliferation markers being confined to the tips of the buds with differentiated cells making up the centers of the enteroids (FIG. 11G). These data indicate that enteroids grown in EREG form a budded morphology with putative crypt like domains budding off of a central lumen surrounded by differentiated cell types, something not previously seen in the standard EGF grown enteroid cultures.

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

1. A method of culturing intestinal cells or tissue, comprising: culturing intestinal cells or tissue in vitro, wherein said culturing comprises culturing in culture medium comprising neuregulin 1 (NRG1).

2. The method of claim 1, wherein said culturing results in the formation of intestinal organoids

3. The method of claim 2, wherein said intestinal organoids are intestinal enteroids.

4. The method of claim 1, wherein said culturing results in stem cell maintenance of said intestinal cells or tissue.

5. The method of claim 1, wherein said cells are stem cells.

6. The method of claim 5, wherein said stem cells are pluripotent stem cells.

7. The method of claim 6, wherein said pluripotent stem cells are induced pluripotent stem cells (iPSCs).

8. The method of claim 1, wherein said intestinal tissue or cells is human intestinal tissue or cells.

9. The method of claim 8, wherein said human intestinal tissue or cells is fetal intestinal tissue.

10. The method of claim 1, wherein said culturing is in Matrigel.

11. The method of claim 1, wherein said culture medium further comprises a R-spondin and Noggin.

12. The method of claim 11, wherein said R-spondin is selected from the group consisting of R-spondin 1, R-spondin 2, and R-spondin 3.

13. The method of claim 1, wherein said culture medium does not comprises EGF.

14. The method of claim 1, wherein said culture medium further comprises Epiregulin (EREG).

15. A composition or kit comprising an intestinal organoid obtained through the method of claim 1.

16. A method of screening a compound, comprising:
a) contacting an intestinal organoid obtained through the method of claim 1 with a test compound; and
b) assaying the effect of the test compound on one or more properties of the intestinal organoid.

17. The method of claim 17, wherein said test compound is a drug.

18. The method of claim 17, wherein said effect is an effect on proliferation of said organoid or toxicity of said test compound.

19. A method of treating an intestinal disease or condition, comprising: implanting an intestinal organoid obtained through the method of claim 1 in the intestine of a subject in need thereof.
